(19) United States
(12) Patent Application Publication
(10) Pub. No.: US 2015/0118177 A1
(43) Pub. Date: Apr. 30, 2015

(54) METHOD FOR PRODUCING A CARE COSMETIC CLEANING AGENT

(71) Applicant: Henkel AG & Co. KGaA, Duesseldorf (DE)

(72) Inventors: Kirsten Sartingen, Brueggen (DE); Heike Schelges, Willich (DE); Barbara Heide, Krefeld (DE); Cecile Guerrera, Toulon (FR)

(21) Appl. No.: 14/593,440

(22) Filed: Jan. 9, 2015

Related U.S. Application Data
(63) Continuation of application No. PCT/EP2013/064227, filed on Jul. 5, 2013.

(30) Foreign Application Priority Data
Jul. 11, 2012 (DE) 10 2012 212 106.4

(51) Int. Cl. A61K 8/81; A61Q 19/10

(52) U.S. Cl. CPC A61K 8/8147; A61Q 19/10
USPC 424/78.02

(57) ABSTRACT

A method for manufacturing a cosmetic cleaning agent includes the steps of a) providing an aqueous dispersion that includes at least one inorganic pigment and/or at least one opacifier and/or luster agent; b) providing a preparation that includes at least one acrylate homo- or copolymer; c) mixing the aqueous dispersion from method step a) with the preparation from method step b); d) providing an O/W emulsion; e) mixing the O/W emulsion from method step d) with the mixture from method step c); f) providing a cosmetically acceptable carrier that includes at least one surfactant; g) mixing the cosmetically acceptable carrier from method step f) with the mixture from method step e).

### FIELD OF THE INVENTION

The present invention generally relates to a method for manufacturing a cleaning agent, and to a cosmetic cleaning agent that includes, besides a pigment and/or an opacifier and/or luster agent, at least one acrylate polymer, an O/W emulsion, and a special surfactant mixture.

### BACKGROUND OF THE INVENTION

Cosmetic cleaning agents have been known for some time and are regularly improved and adapted to consumers' changing needs.

For example, consumers expect not only good cleaning and refreshment from a modern cleaning agent, but also care-providing properties. In particular, cleaning agents are intended not to leave behind on the skin a feeling of stress and/or dryness after being used. The skin should instead feel soft, smooth, and moisturized after cleaning.

The visual and haptic impression of a cleaning agent also plays an increasingly important role, with the result that the manufacture of products having a rich, creamy consistency is particularly desirable.

It is known to add care-providing oils and/or waxes to cosmetic cleaning agents in order to improve skin care.

The manufacture of such care-providing cleaning agents is problematic, however, since on the one hand the stabilization of larger quantities of oil and/or wax (which are desirable in order to obtain a sufficient care-providing effect) often presents considerable difficulties; and on the other hand larger quantities of oils and/or waxes in cosmetic cleaning agents have a disadvantageous effect on the foaming properties (in particular the foam volume) of the agents.

A further problem is that given the usual rather short residence time of a cleaning agent on the skin, only small quantities of care-providing components can be deposited onto the skin before most of the care-providing components are rinsed off again.

A demand therefore continues to exist for care-providing cosmetic cleaning agents that are accessible via a simple manufacturing method and offer a conditioning advantage for the skin.

It is therefore desirable to provide an uncomplicated method for manufacturing a care-providing cleaning agent, in particular a care-providing skin cleaning agent.

Such a cleaning agent should include an effective quantity of at least one care-providing oil without necessitating additional complex and energy-intensive steps in order to stabilize the care-providing oil in the context of the manufacture and/or storage of the cleaning agent.

The cleaned skin should feel clean, supple, and smooth after utilization of the agents.

It is further desirable to manufacture care-providing cosmetic cleaning agents that have a lotion-like appearance and feel.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

According to one aspect of the invention, a method for manufacturing a cosmetic cleaning agent includes the following steps: a) providing an aqueous dispersion that includes at least one inorganic pigment and/or at least one opacifier and/or luster agent, b) providing a preparation that includes at least one acrylate homo- or copolymer, c) mixing the aqueous dispersion from method step a) with the preparation from method step b), d) providing an O/W emulsion, e) mixing the O/W emulsion from method step d) with the mixture from method step c), f) providing a cosmetically acceptable carrier that includes at least one surfactant, and g) mixing the cosmetically acceptable carrier from method step f) with the mixture from method step e).

According to another aspect of the invention, a cosmetic cleaning agent includes 0.01 to 5 wt % of at least one inorganic pigment and/or at least one opacifier and/or luster agent; 0.01 to 10 wt % of at least one acrylate homo- or copolymer; 0.1 to 15 wt % of at least one O/W emulsion that includes (i) at least one fatty alcohol and/or at least one ethoxylated fatty alcohol of the following formula (I)

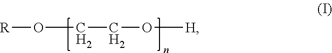

in which R denotes a straight-chain or branched, saturated or unsaturated alkyl residue having 10 to 22 carbon atoms, and the index n denotes the number 0 or a number from 5 to 25, (ii) glycerol and/or at least one glycerol mono- and/or diester of glycerol and at least one straight-chain or branched, saturated or unsaturated carboxylic acid that has a carbon chain length from 10 to 22 carbon atoms, and (iii) at least one fatty acid ester of the following formula (II)

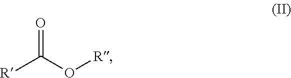

in which R′ denotes a straight-chain or branched, saturated or unsaturated alkyl residue having 5 to 18 carbon atoms, and R″ denotes a straight-chain or branched, saturated or unsaturated alkyl residue having 10 to 22 carbon atoms; 3 to 20 wt % of at least one anionic surfactant; and 0.1 to 15 wt % of at least one amphoteric, zwitterionic, or nonionic surfactant, wherein the quantity indications refer to the total weight of the cleaning agent.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the invention is a method for manufacturing a cosmetic cleaning agent, which comprises the following steps:
a) providing an aqueous dispersion that includes at least one inorganic pigment and/or at least one opacifier and/or luster agent,
b) providing a preparation that includes at least one acrylate homo- or copolymer,
c) mixing the aqueous dispersion from method step a) with the preparation from method step b),
d) providing an O/W emulsion,
e) mixing the O/W emulsion from method step d) with the mixture from method step c),
f) providing a cosmetically acceptable carrier that includes at least one surfactant,
g) mixing the cosmetically acceptable carrier from method step f) with the mixture from method step e).

The inorganic pigment and/or the opacifier and/or luster agent is/are prepared in method step a) as an aqueous dispersion. The mixing ratio of water to inorganic pigment and/or opacifier and/or luster agent is preferably in the range from 30:1 to 1:1, particularly preferably in the range from 20:1 to 5:1.

In order to achieve the visual “lotion” character of the cleaning agent, it is preferred if the concentration of inorganic pigment(s) and/or opacifier and/or luster agent is 0.01 to 5 wt %, preferably 0.025 to 3 wt %, and in particular 0.05 to 2.5 wt %, wherein the quantity indications refer to the total weight of the cosmetic cleaning agent.

“Suitable inorganic pigments” are understood preferably as pigments that have a refractive index >1.8, more preferably >1.9, and in particular >2.0.

Examples of particularly suitable inorganic pigments are the so-called white pigments such as titanium dioxide, lithopone, zinc oxide, zinc sulfide, and/or calcium carbonate. Titanium dioxide, which is prepared in method step a) particularly preferably as a 10- to 20-% dispersion in water, is especially preferred.

Suitable luster agents and/or opacifiers are to be understood, for example, as:
mono- and/or diesters of ethylene glycol, of 1,2-propanediol, of glycerol, and/or of a polyethylene glycol with at least one C₈ to C₂₄ carboxylic acid, and/or
styrene/acrylate copolymers.

The opacifiers and/or luster agents known by the following INCI names are particularly suitable:

Glycol Distearate, for example the commercial product Cutina® AGS of the Cognis company, Glycol Monostearate, for example the commercial product Cutina® EGMS of the Cognis company, PEG-3 Distearate, for example the commercial product Genapol® TS of the Clariant company, PEG-2 Distearate, for example the commercial product Kessco® DEGMS of the Akzo Nobel company, Propylene Glycol Stearate, for example the commercial product Tegin® P of the Goldschmidt company, and/or Styrene/Acrylates Copolymers, for example the commercial products Joncryl® 67 of the Johnson Polymers company, Suprawal® WS of the BASF company, and/or Acusol® OP 301 of the Rohm & Haas company.

The opacifiers and/or luster agents known by the following INCI names are particularly suitable for use in the manufacturing method:

Glycol Distearate, Glycol Monostearate, PEG-3 Distearate, and/or Styrene/Acrylates Copolymer.

In method step b), firstly a preparation which includes at least one acrylate homo- or copolymer is provided. A “preparation” is understood both as the respective pure polymer powder and as a solution, dispersion, or emulsion of the respective polymer, preferably in water.

The solution, dispersion, or emulsion of the respective polymer can of course also include further usual ingredients, for example preservatives.

In a preferred embodiment, the acrylate homo- or copolymer is provided as an aqueous solution, dispersion, or emulsion, and in method step c) is mixed with the aqueous dispersion from method step a).

The sequence of mixing the preparations from method steps a) and b) in method step c) can occur in any way (a) to b) or b) to a)).

In a preferred embodiment, however, the aqueous dispersion a) is added to the preparation b).

The mixing in method step c) occurs preferably at room temperature, and the result thereof is preferably a viscous, milky preparation.

In a further preferred embodiment it can be advantageous to adjust the pH of the mixture from method step c) to a value in the range from approximately 4 to 7, preferably to a value in the range from 5 to 7, and in particular to a value in the range from 6 to 7.

Suitable acrylate homo- and copolymers are to be understood as preferably crosslinked or uncrosslinked polyacrylates and/or crosslinked or uncrosslinked copolymers of (meth)acrylic acid with at least one (meth)acrylic acid ester.

The polymers are preferably anionic ones, which optionally can be hydrophobically modified.

Examples of anionic monomers from which the acrylate homo- and copolymers can be made are acrylic acid, methacrylic acid, crotonic acid, itaconic acid, maleic acid anhydride, and 2-acrylamido-2-methylpropanesulfonic acid. The acid groups can be present entirely or partly as a sodium, potassium, ammonium, or mono- or triethanolammonium salt. Preferred monomers are 2-acrylamido-2-methylpropanesulfonic acid and (meth)acrylic acid.

Preferred anionic homopolymers are uncrosslinked and crosslinked polyacrylic acids. Allyl ethers of pentaerythritol, of sucrose, and of propylene can be preferred crosslinking agents. Such compounds are obtainable commercially, for example, under the trademark Carbopol®.

The homopolymer of 2-acrylamido-2-methylpropanesulfonic acid that is obtainable commercially, for example, under the designation Rheothik® 11-80 is likewise preferred.

Also preferred are uncrosslinked and crosslinked, hydrophobically modified polyacrylic acids that are obtainable as approximately 30-wt % emulsions in water, for example under the commercial designations Carbopol® Aqua SF1, Carbopol® Aqua SF2, or Rheomer® 33, from a variety of suppliers.

Preferred anionic acrylate copolymers are to be understood as copolymers of at least one anionic monomer and at least one nonionogenic monomer. The reader is referred to the substances discussed above with regard to the anionic monomers. Preferred nonionogenic monomers are acrylamide, methacrylamide, acrylic acid esters, methacrylic acid esters, itaconic acid mono- and diesters, vinylpyrrolidinone, vinyl ethers, and vinyl esters.

Preferred anionic copolymers are, for example, copolymers of acrylic acid, methacrylic acid, and/or C₁ to C₆ alkyl esters thereof, such as those marketed under the INCI declaration Acrylates Copolymer. Preferred commercial products are, for example, Aculyn® 33 of the Rohm & Haas company and/or Rheocare® TTA of the Cognis company. Also preferred, however, are copolymers of acrylic acid, methacrylic acid, or C₁ to C₆ alkyl esters thereof and esters of an ethylenically unsaturated acid and an alkoxylated fatty alcohol. Suitable ethylenically unsaturated acids are, in particular, acrylic acid, methacrylic acid, and itaconic acid; suitable alkoxylated fatty alcohols are, in particular, Steareth-20 or Ceteth-20. Copolymers of this kind are marketed by the Rohm & Haas company under the commercial designation Aculyn® 22 and by the National Starch company under the commercial designations Structure® 2001 and Structure® 3001.

It has been found that aqueous emulsions or dispersions of the aforementioned crosslinked or uncrosslinked, hydrophobically modified polyacrylates and/or crosslinked or uncrosslinked (meth)acrylic acid-(meth)acrylic acid ester copolymers are particularly suitable for use in the method according to the present invention, since even at room temperature and at pH values in the slightly acidic to neutral pH range, they already form gel networks that not only allow greater quantities of the O/W emulsion can be stabilized over a long period of time in the cleaning agent, but at the same time cause the pigments, luster agents, and/or opacifiers that are insoluble in the cleaning agent to remain suspended in stable fashion.

In method step d), an O/W emulsion is provided.

The O/W emulsions suitable for the method according to the present invention usually include at least one oil, water, and at least one emulsifier agent. They are manufactured with usual methods known in the existing art.

Alternatively, a prefabricated, commercially obtainable emulsion can also be provided in method step d) as a suitable O/W emulsion.

The O/W emulsion of method step d) is used in the method according to the present invention preferably in a quantity from 0.1 to 20 wt %, preferably in a quantity from 0.2 to 15 wt %, and in particular in a quantity from 0.3 to 10 wt %, wherein the quantity indications refer to the total weight of the cleaning agent.

Suitable oils for manufacturing the O/W emulsions can be selected from mineral, natural, and synthetic oil components, and/or from fatty substances.

Natural (vegetable) oils that can be used are triglycerides and mixtures of triglycerides. Preferred natural oils are coconut oil, (sweet) almond oil, walnut oil, peach kernel oil, apricot kernel oil, avocado oil, tea tree oil, soy oil, sesame oil, sunflower oil, tsubaki oil, evening primrose oil, rice bran oil, palm kernel oil, mango kernel oil, lady's smock oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter, and shea butter.

Mineral oils used are in particular mineral oil, paraffin and isoparaffin oils, and synthetic hydrocarbons. An example of a usable hydrocarbon is, for example, 1,3-di-(2-ethylhexyl)cyclohexane, obtainable as a commercial product (Cetiol® S).

Silicone compounds are suitable as synthetic oils.

Suitable silicones can be selected from among:
(i) polyalkylsiloxanes, polyarylsiloxanes, polyalkylarylsiloxanes, which are volatile or nonvolatile, straight-chain, branched, or cyclic, crosslinked or not crosslinked;
(ii) polysiloxanes that include in their general structure one or more organofunctional groups that are selected from among:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and
   m) sulfate or thiosulfate groups;
(iii) linear polysiloxane (A)-polyoxyalkylene(B) block copolymers of the (A-B)ₙ type where n>3;
(iv) grafted silicone polymers having a non-silicone-containing organic backbone, which are made of an organic main chain that is constituted from organic monomers that include no silicone, onto which at least one polysiloxane macromere has been grafted in the chain and optionally at at least one chain end;
(v) grafted silicone polymers having a polysiloxane backbone onto which non-silicone-containing organic monomers have been grafted, which monomers comprise a polysiloxane main chain onto which at least one organic macromere that includes no silicone has been grafted in the chain and optionally at at least one of its ends;
(vi) or mixtures thereof.

A dialkyl ether can furthermore serve as an oil component.

Usable dialkyl ethers are in particular di-n-alkyl ethers having in total between 12 and 36 carbon atoms, in particular 12 to 24 carbon atoms, for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as di-tert-butyl ether, diisopentyl ether, di-3-ethyldecyl ether, tert-butyl-n-octyl ether, isopentyl-n-octyl ether, and 2-methylpentyl-n-octyl ether.

Di-n-octyl ether, which is obtainable commercially under the name Cetiol® CE, is particularly preferred.

“Fatty substances” are to be understood as fatty acids, fatty alcohols, and natural and synthetic waxes, which can be present both in solid form and in liquid form in aqueous dispersion.

Fatty acids that can be used are linear and/or branched, saturated and/or unsaturated fatty acids having 6 to 30 carbon atoms. Fatty acids having 10 to 22 carbon atoms are preferred. Among those that might be recited are, for example, isostearic acids, such as the commercial products Emersol® 871 and Emersol® 875, and isopalmitic acids such as the commercial product Edenor® IP 95, as well as all further fatty acids marketed under the Edenor® commercial designations (Cognis). Further typical examples of such fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof.

The fatty acid cuts that are obtainable from coconut oil or palm oil are usually particularly preferred; the use of stearic acid is, as a rule, particularly preferred.

Fatty alcohols that can be used are saturated, mono- or polyunsaturated, branched or unbranched fatty alcohols having C₆ to C₃₀, preferably C₁₀ to C₂₂, and very particularly preferably C₁₂ to C₂₂ carbon atoms. For example, decanol, octanol, octenol, dodecenol, decenol, octadienol, dodecadienol, decadienol, oleyl alcohol, erucyl alcohol, ricinol alcohol, stearyl alcohol, isostearyl alcohol, cetyl alcohol, lauryl alcohol, myristyl alcohol, arachidyl alcohol, capryl alcohol, caprinyl alcohol, linoleyl alcohol, linolenyl alcohol, and behenyl alcohol, as well as Guerbet alcohols thereof, are usable, this listing being intended to be exemplary and not limiting in nature. The fatty alcohols derive, however, from preferably natural fatty acids; it is usually possible to proceed from an extraction from the esters of the fatty acids by reduction. Also usable according to the present invention are those fatty alcohol cuts which are generated by the reduction of naturally occurring triglycerides such as beef tallow, palm oil, peanut oil, colza oil, cottonseed oil, soy oil, sunflower oil, and linseed oil, or from fatty acid esters resulting from transesterification products thereof with corresponding alcohols, and thus represent a mixture of different fatty alcohols. Such substances are available commercially, for example, under the designations Stenol®, e.g. Stenol® 1618, or Lanette®, e.g. Lanette® O, or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16, or Isocarb® 24. It is of course also possible according to the present invention to use wool-wax alcohols such as those available commercially under the designations Corona®, White Swan®, Coronet®, or Fluilan®.

Natural or synthetic waxes that can be used are solid paraffins or isoparaffins, carnauba waxes, beeswaxes, candelilla waxes, ozocerites, ceresin, spermaceti, sunflower wax, fruit waxes such as apple wax or citrus wax, microcrystalline waxes made from PE or PP. Such waxes are obtainable, for example, via Kahl & Co., Trittau.

Further suitable fatty substances are, for example:
Ester oils. “Ester oils” are to be understood as esters of C₆ to C₃₀ fatty acids, in particular of C₅ to C₁₈ fatty acids, with C₂ to C₃₀ fatty alcohols, in particular with C₁₀ to C₂₂ fatty alcohols. Examples of fatty-acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof.
Examples of fatty-alcohol components in the ester oils are isopropyl alcohol, capronyl alcohol, capryl alcohol, 2-ethylhexyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol, as well as industrial mixtures thereof Isopropyl myristate (Rilanit® IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, cetyl palmitate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred.
dicarboxylic acid esters such as di-n-butyl adipate, di-(2-ethylhexyl)adipate, di-(2-ethylhexyl)succinate, and diisotridecyl acelaate, as well as diol esters such as ethylene glycol dioleate, ethylene glycol diisotridecanoate, propylene glycol di-(2-ethylhexanoate), propylene glycol diisostearate, propylene glycol dipelargonate, butanediol diisostearate, neopentyl glycol dicaprylate,
symmetrical, asymmetrical, or cyclic esters of carbonic acid with fatty alcohols,
glycerol carbonate or dicaprylyl carbonate (Cetiol® CC),
ethoxylated or non-ethoxylated fatty acid mono-, di- and triesters of saturated and/or unsaturated linear and/or branched fatty acids with glycerol, for example the commercial products Tegin® M, Monomuls® 90-O18, Monomuls® 90-L12, Cetiol® HE, or Cutina® MD.

In a preferred embodiment the O/W emulsion includes at least one natural oil and/or at least one of the fatty acid esters recited above and/or at least one of the glycerol mono- or diesters recited above and/or at least one of the fatty alcohols recited above.

Preferably 0.5 to 50 wt %, more preferably 1 to 45, and in particular 5 to 40 wt % of one or more of the oils recited above is used to manufacture suitable O/W emulsions, wherein the quantity indications refer to the total weight of the O/W emulsion.

A further essential ingredient of the O/W emulsion in method step d) is water. The O/W emulsion includes, based on its total weight, preferably 30 to 90 wt %, more preferably 40 to 80 wt %, and in particular 45 to 75 wt % water.

Suitable emulsifier agents are used in the O/W emulsions (based on their total weight) preferably in a quantity from 0.1 to 50 wt %, more preferably from 0.5 to 40 wt %, and in particular from 1 to 35 wt %, and can preferably be selected from:
C₈ to C₃₀ fatty acid mono- and diesters of addition products of 1 to 30 mol ethylene oxide with glycerol,
addition products of 2 to 50 mol ethylene oxide and/or 0 to 5 mol propylene oxide with linear and branched fatty alcohols having 8 to 30 carbon atoms or with fatty acids having 8 to 30 carbon atoms and with alkyl phenols having 8 to 15 carbon atoms in the alkyl group,
sorbitan fatty acid esters and addition products of ethylene oxide with sorbitan fatty acid esters, for example polysorbates,
sugar fatty acid esters and addition products of ethylene oxide with sugar fatty acid esters,
addition products of ethylene oxide with fatty acid alkanolamides and fatty amines and/or alkyl polyglucosides.

In a preferred embodiment the O/W emulsions from method step d) include at least one emulsifier agent from the group of addition products of 2 to 50 mol ethylene oxide and/or 0 to 5 mol propylene oxide with linear and branched fatty alcohols having 8 to 30 carbon atoms. C₁₂ to C₂₀ fatty alcohols having a degree of ethoxylation from 10 to 25 are more preferred.

In a particularly preferred embodiment, in method step d) an O/W emulsion is provided which includes, besides water,
(i) at least one fatty alcohol and/or at least one ethoxylated fatty alcohol of the following formula (I)

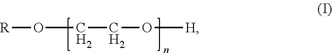

in which R denotes a straight-chain or branched, saturated or unsaturated alkyl residue having 10 to 22 carbon atoms, and the index n denotes the number 0 or a number from 5 to 25,
(ii) glycerol and/or at least one glycerol mono- and/or diester of glycerol and at least one straight-chain or branched, saturated or unsaturated carboxylic acid that has a carbon chain length from 10 to 22 carbon atoms,
(iii) at least one fatty acid ester of the following formula (II)

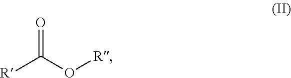

in which R′ denotes a straight-chain or branched, saturated or unsaturated alkyl residue having 5 to 18 carbon atoms, and R″ denotes a straight-chain or branched, saturated or unsaturated alkyl residue having 10 to 22 carbon atoms.

Within this embodiment it is particularly preferred if the O/W emulsion in method step d) includes
(i) at least one fatty alcohol and at least one ethoxylated fatty alcohol in accordance with formula (I),
(ii) glycerol and at least one glycerol mono- and/or diester, and
(iii) at least two different carboxylic acid esters of formula (II).

An example of a particularly preferred commercially obtainable O/W emulsion is the O/W emulsion obtainable under the name Emulgade® CM.

In method step e) the O/W emulsion from method step d) is mixed with the mixture from method step c). The mixing sequence can be arbitrary, and preferably occurs at room temperature.

Usually, however, the O/W emulsion from method step d) is added, with gentle stirring, to the mixture from method step c).

In method step f) a cosmetically acceptable carrier that includes at least one surfactant is provided.

A “cosmetically acceptable carrier” is understood preferably as an aqueous or aqueous alcoholic carrier.

The cosmetic carrier preferably includes at least 40 wt % water.

The cosmetic carrier can furthermore include 0.01 to 40 wt %, preferably 0.05 to 35 wt %, and in particular 0.1 to 30 wt % of at least one alcohol that can be selected from ethanol, ethyl diglycol, 1-propanol, 2-propanol, isopropanol, 1,2-propylene glycol, glycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, sorbitol, benzyl alcohol, phenoxyethanol, or mixtures of these alcohols.

Water-soluble alcohols are preferred.

Ethanol, ethyl diglycol, 1-propanol, 2-propanol, isopropanol, 1,2-propylene glycol, glycerol, benzyl alcohol, and/or phenoxyethanol, as well as mixtures of these alcohols, are particularly preferred.

Surfactants suitable for the method according to the present invention can be selected from high-foaming mild anionic, amphoteric/zwitterionic, and/or nonionic surfactants. In order to achieve optimum mildness and avoid or reduce drying of the skin during cleaning, it is advantageous if a mixture of mild anionic and mild amphoteric surfactants in an above-described carrier is provided in method step f).

The total surfactant content in the cleaning agent is preferably a maximum of 16 wt %, more preferably a maximum of 15 wt %, particularly preferably a maximum of 14 wt %, and particularly preferably a maximum of 12.5 wt % (based on the total weight of the cleaning agent).

Suitable anionic surfactants can be used in the method according to the present invention preferably in quantities from 0.1 to 30 wt %, more preferably from 0.5 to 27.5 wt %, particularly preferably from 1 to 25 wt %, and in particular from 3 to 20 wt %, wherein the quantity indications refer to the total weight of the cleaning agent.

Suitable anionic surfactants include:
linear and branched fatty acids having 8 to 30 carbon atoms (soaps);
ether carboxylic acids of the formula R—O—(CH₂—CH₂O)ₓ—CH₂—COOH, in which R is a linear or branched, saturated or unsaturated alkyl group having 8 to 30 carbon atoms and x=0 or is 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid mono- and/or diallyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups;
alpha-olefinsulfonates having 8 to 24 carbon atoms;
alkyl sulfate salts and/or alkyl polyglycol ether sulfate salts of the formula R—O(CH₂—CH₂O)—OSO₃⁻X⁺ in which R is a preferably linear or branched, saturated or unsaturated alkyl group having 8 to 30 carbon atoms, x=0 or is 1 to 12, and X is an alkali ion or ammonium ion;
sulfonates of unsaturated fatty acids having 8 to 24 carbon atoms and 1 to 6 double bonds;
esters of tartaric acid and citric acid with alcohols which represent addition products of approximately 2 to 15 molecules of ethylene oxide and/or propylene oxide with fatty alcohols having 8 to 22 carbon atoms;
alkyl and/or alkenyl ether phosphates of the formula

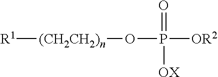

in which R¹ preferably denotes an aliphatic hydrocarbon residue having 8 to 30 carbon atoms, R² denotes hydrogen, a (CH₂CH₂Oₙ)R¹ residue, or X, n denotes numbers from 0 to 10, and X denotes hydrogen, an alkali metal or alkaline earth metal, or NR³R⁴R⁵R⁶, where R³ to R⁶ mutually independently denote a C₁ to C₄ hydrocarbon residue.

Preferred anionic surfactants are ether carboxylic acids of the formula recited above, acyl sarcosides having 8 to 24 carbon atoms in the acyl group, sulfosuccinic acid mono- and/or diallyl esters having 8 to 24 carbon atoms in the alkyl group and sulfosuccinic acid monoalkylpolyoxyethyl esters having 8 to 24 carbon atoms in the alkyl group and 1 to 6 oxyethyl groups, alpha-olefinsulfonates having 8 to 24 carbon atoms, and/or alkyl sulfate salts and/or alkyl polyglycol ether sulfate salts of the formula recited above.

Particularly preferred anionic surfactants are straight-chain or branched alkyl ether sulfates that include an alkyl residue having 8 to 18 and in particular having 10 to 16 carbon atoms as well as 1 to 6 and in particular 2 to 4 ethylene oxide units.

Further particularly preferred anionic surfactants are straight-chain or branched alkylsulfonates that include an alkyl residue having 8 to 18 and in particular having 10 to 16 carbon atoms. The sodium, magnesium, and/or triethanolamine salts of linear or branched lauryl, tridecyl, and/or myristyl sulfates, which have a degree of ethoxylation from 2 to 4, are particularly preferred.

Suitable amphoteric/zwitterionic surfactants can be used in the method according to the present invention preferably in quantities from 0.01 to 25 wt %, more preferably from 0.05 to 20 wt %, particularly preferably from 0.075 to 17.5 wt %, and in particular from 0.1 to 15 wt %, wherein the quantity indications refer to the total weight of the cleaning agent.

Suitable amphoteric/zwitterionic surfactants can be selected from compounds of the following formulas (i) to (v), in which the residue R respectively denotes a straight-chain or branched, saturated or mono- or polyunsaturated alkyl or alkenyl residue having 8 to 24 carbon atoms:

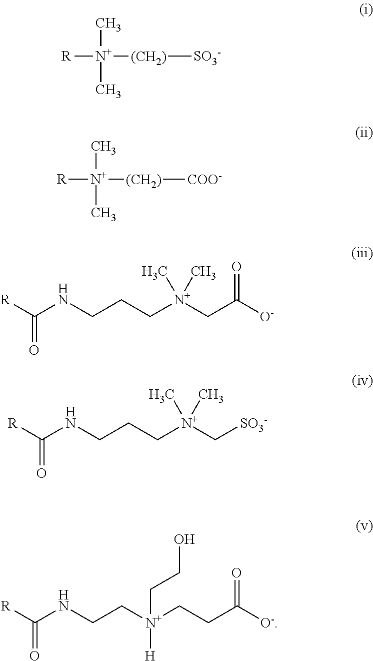

Particularly suitable amphoteric/zwitterionic surfactants are alkylamidoalkyl betaines and/or alkylampho(di)acetates of formulas (i) to (v) recited above.

Including among the particularly suitable amphoteric/zwitterionic surfactants are the surfactants known by the INCI names Cocamidopropyl Betaine and Disodium Cocoamphodiacetate.

Suitable nonionic surfactants can be used in the method according to the present invention preferably in quantities from 0 to 20 wt %, more preferably from 0.25 to 17.5 wt %, particularly preferably from 0.5 to 15 wt %, and in particular from 1 to 10 wt %, wherein the quantity indications refer to the total weight of the conditioning cleaning agent.

Included among the suitable nonionic surfactants/emulsifying agents are those nonionic surfactants and emulsifier agents that have already been recited at an earlier point in the description.

For the case in which a nonionic surfactant is present in the cosmetic carrier, alkyl oligoglucosides, in particular alkyl oligoglucosides based on hardened C_{12/14} coconut alcohol having a DP from 1 to 3, such as those obtainable commercially e.g. under the INCI name Coco Glucoside, are preferred.

Further preferred nonionic surfactants that can be included in the cosmetic carrier are C₈ to C₃₀ fatty acid mono- and diesters of addition products of 1 to 30 mol ethylene oxide with glycerol. The C₁₀ to C₁₆ fatty acid mono- and diesters of addition products of 1 to 10 mol ethylene oxide with glycerol are particularly preferred. The one known by the INCI name PEG-7 Glyceryl Cocoate is especially preferred.

In a particularly preferred embodiment

Suitable protein hydrolysates are preferably of vegetable, animal, or marine origin and are used in the method according to the present invention preferably in a quantity from 0.01 to 10 wt %, more preferably from 0.25 to 7.5 wt %, and in particular in a quantity from 0.05 to 5 wt %, wherein the quantity indications refer to the total weight of the conditioning cleaning agent.

Suitable animal protein hydrolysates are, for example, elastin, collagen, keratin, silk, and/or milk protein hydrolysates, which can also be present in the form of salts.

Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Suitable protein hydrolysates of vegetable origin are, for example, soy, almond, rice, pea, potato, canola, and/or wheat protein hydrolysates.

Such products are obtainable, for example, under the trademarks Gluadin® (Cognis), DiaMin® (Diamalt), Lexein® (Inolex), and Crotein® (Croda).

Among the suitable protein hydrolysates of marine origin are, for example, collagen hydrolysates from fish or algae, as well as protein hydrolysates from mussels or pearl hydrolysates. Examples of suitable pearl hydrolysates are the commercial products Pearl Protein Extract BG® or Crodarom® Pearl.

Cationized protein hydrolysates are also usable, wherein the underlying protein hydrolysate can derive from the above-described animal, vegetable, and/or marine sources.

Quaternized amino acids and mixtures thereof are also to be understood as cationic protein hydrolysates. Quaternization of the protein hydrolysates or of the amino acids is often carried out by means of quaternary ammonium salts such as N,N-dimethyl-N-(n-alkyl)-N-(2-hydroxy-3-chloro-n-propyl)ammonium halides.

The cationic protein hydrolysates can also be even further derivatized.

Typical examples of suitable cationic protein hydrolysates and/or derivatives that may be recited are the products known by the following INCI names and obtainable commercially: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

Protein hydrolysates from animal sources, in particular elastin, collagen, keratin, and/or silk protein hydrolysates, which preferably have an average molecular weight (weight average) from 100 to 2500, more preferably from 200 to 2000, particularly preferably from 300 to 1500, and in particular from 400 to 1200 dalton, are particularly preferred for use in the method according to the present invention.

Suitable commercial products are obtainable, for example, from the Croda company under the designation ProSina®.

Surfactants suitable for the method according to the present invention can be selected from high-foaming mild anionic, amphoteric/zwitterionic, and/or nonionic surfactants.

Suitable anionic surfactants can be used in the method according to the present invention preferably in quantities from 0.01 to 40 wt %, more preferably from 0.05 to 30 wt %, particularly preferably from 1 to 25 wt %, and in particular from 3 to 20 wt %, wherein the quantity indications refer to the total weight of the conditioning cleaning agent.

Suitable anionic surfactants include:
linear and branched fatty acids having 8 to 30 carbon atoms (soaps);
ether carboxylic acids of the formula R—O—(CH₂—CH₂O)ₓ—CH₂—COOH, in which R is a linear or branched, saturated or unsaturated alkyl group having 8 to 30 carbon atoms and x=0 or is 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid mono- and/or dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups;
alpha-olefinsulfonates having 8 to 24 carbon atoms;
alkyl sulfate salts and/or alkyl polyglycol ether sulfate salts of the formula R—O(CH₂—CH₂O)—OSO₃⁻X⁺ in which R is a preferably linear or branched, saturated or unsaturated alkyl group having 8 to 30 carbon atoms, x=0 or is 1 to 12, and X is an alkali ion or ammonium ion;
sulfonates of unsaturated fatty acids having 8 to 24 carbon atoms and 1 to 6 double bonds;
esters of tartaric acid and citric acid with alcohols which represent addition products of approximately 2 to 15 molecules of ethylene oxide and/or propylene oxide with fatty alcohols having 8 to 22 carbon atoms;
alkyl and/or alkenyl ether phosphates of the formula

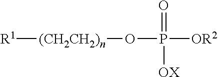

in which R¹ preferably denotes an aliphatic hydrocarbon residue having 8 to 30 carbon atoms, R² denotes hydrogen, a (CH₂CH₂Oₙ)R¹ residue, or X, n denotes numbers from 0 to 10, and X denotes hydrogen, an alkali metal or alkaline earth metal, or NR³R⁴R⁵R⁶, where R³ to R⁶ mutually independently denote a C₁ to C₄ hydrocarbon residue.

Preferred anionic surfactants are ether carboxylic acids of the formula recited above, acyl sarcosides having 8 to 24 carbon atoms in the acyl group, sulfosuccinic acid mono- and/or dialkyl esters having 8 to 24 carbon atoms in the alkyl group and sulfosuccinic acid monoalkylpolyoxyethyl esters having 8 to 24 carbon atoms in the alkyl group and 1 to 6 oxyethyl groups, alpha-olefinsulfonates having 8 to 24 carbon atoms, and/or alkyl sulfate salts and/or alkyl polyglycol ether sulfate salts of the formula recited above.

Particularly preferred anionic surfactants are straight-chain or branched alkyl ether sulfates that include an alkyl residue having 8 to 18 and in particular having 10 to 16 carbon atoms as well as 1 to 6 and in particular 2 to 4 ethylene oxide units.

Further particularly preferred anionic surfactants are straight-chain or branched alkylsulfonates that include an alkyl residue having 8 to 18 and in particular having 10 to 16 carbon atoms. The sodium, magnesium, and/or triethanolamine salts of linear or branched lauryl, tridecyl, and/or myristyl sulfates, which have a degree of ethoxylation from 2 to 4, are particularly preferred.

Suitable amphoteric/zwitterionic surfactants can be used in the method according to the present invention preferably in quantities from 0 to 20 wt %, more preferably from 0.25 to 17.5 wt %, particularly preferably from 0.5 to 15 wt %, and in particular from 1 to 10 wt %, wherein the quantity indications refer to the total weight of the conditioning cleaning agent.

Suitable amphoteric/zwitterionic surfactants can be selected from compounds of the following formulas (i) to (v), in which the residue R respectively denotes a straight-chain or branched, saturated or mono- or polyunsaturated alkyl or alkenyl residue having 8 to 24 carbon atoms:

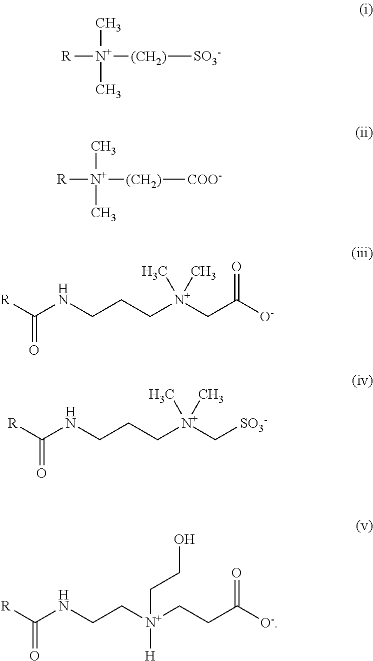

Particularly suitable amphoteric/zwitterionic surfactants are alkylamidoalkyl betaines and/or alkylampho(di)acetates of formulas (i) to (v) recited above.

Including among the particularly suitable amphoteric/zwitterionic surfactants are the surfactants known by the INCI names Cocamidopropyl Betaine and Disodium Cocoamphodiacetate.

Suitable nonionic surfactants can be used in the method according to the present invention (in addition to the alkyl oligoglycoside(s) in the microemulsion a)) preferably in quantities from 0 to 20 wt %, more preferably from 0.25 to 17.5 wt %, particularly preferably from 0.5 to 15 wt %, and in particular from 1 to 10 wt %, wherein the quantity indications refer to the total weight of the conditioning cleaning agent.

Included among the suitable nonionic surfactants/,emulsifier agents are, for example:
C₈ to C₃₀ fatty acid mono- and diesters of addition products of 1 to 30 mol ethylene oxide with glycerol,
amine oxides,
addition products of 2 to 50 mol ethylene oxide and/or 0 to 5 mol propylene oxide with linear and branched fatty alcohols having 8 to 30 carbon atoms, with fatty acids having 8 to 30 carbon atoms, and with alkyl phenols having 8 to 15 carbon atoms in the alkyl group,
sorbitan fatty acid esters and addition products of ethylene oxide with sorbitan fatty acid esters, for example polysorbates,
sugar fatty acid esters and addition products of ethylene oxide with sugar fatty acid esters,
addition products of ethylene oxide with fatty acid alkanolamides and fatty amines, and/or
alkyl polyglucosides.

For the case in which a nonionic surfactant is used as a further surfactant in the method according to the present invention, alkyl oligoglucosides, in particular alkyl oligoglucosides based on hardened C_{12/14} coconut alcohol having a DP from 1 to 3, such as those obtainable commercially e.g. under the INCI name Coco Glucoside, are preferred.

Further preferred nonionic surfactants are C₈ to C₃₀ fatty acid mono- and diesters of addition products of 1 to 30 mol ethylene oxide with glycerol. The C₁₀ to C₁₆ fatty acid mono- and diesters of addition products of 1 to 10 mol ethylene oxide with glycerol are particularly preferred. The one known by the INCI name PEG-7 Glyceryl Cocoate is especially preferred.

In a particularly preferred embodiment the cosmetic carrier in method step f) includes
a. 3 to 20 wt % of at least one anionic surfactant, and
b. 0.1 to 15 wt % of at least one amphoteric, zwitterionic, or nonionic surfactant,
   wherein the quantity indications refer to the total weight of the cleaning agent.

Lastly, in method step g) the cosmetic carrier from method step f) is mixed with the preparation from method step e).

Mixing occurs preferably at room temperature with gentle stirring.

It is possible both to add the mixture from method step e) to the carrier from method step f), and to add the carrier from method step f) to the mixture from method step e).

Method step g) is optionally followed by a further step in which the pH and/or viscosity of the cleaning agent can additionally be slightly varied.

In order to assist deposition of the O/W emulsion or of the care-providing component(s) from the O/W emulsion onto the skin, it can be advantageous if the cosmetic carrier in method step f) furthermore includes at least cationic deposition polymer.

The presence of at least one cationic polymer in the cosmetic carrier is furthermore preferred, since cationic polymers also have a skin-conditioning effect.

Suitable cationic polymers can be added to the cosmetic carrier preferably in a quantity from 0.01 to 20 wt %, more preferably in a quantity from 0.05 to 15 wt %, and in particular in a quantity from 0.1 to 10 wt %.

The resulting cosmetic cleaning agent includes, based on its total weight, preferably 0.05 to 10 wt %, more preferably 0.1 to 5 wt %, and in particular 0.2 to 3 wt % of at least one cationic polymer.

Suitable cationic polymers are, for example:
quaternized cellulose derivatives such as those commercially obtainable under the names Celquat® and Polymer JR®,
hydrophobically modified cellulose derivatives, for example the cationic polymers marketed under the commercial name SoftCat®,
cationic alkyl polyglycosides,
cationized honey, for example the commercial product Honeyquat® 50,
cationic guar derivatives, such as in particular the products marketed under the commercial names Cosmedia® Guar and Jaguar®,
polymeric dimethyldiallylammonium salts and copolymers thereof with esters and amides of acrylic acid and methacrylic acid. The products obtainable commercially under the designations Merquat® 100 (poly(dimethyldiallylammonium)chloride) and Merquat 550 (dimethyldiallylammonium chloride/acrylamide copolymer) are examples of such cationic polymers,
copolymers of vinylpyrrolidone with quaternized derivatives of dialkylaminoacrylate and -methacrylate, for example vinylpyrrolidone/dimethylaminoethyl methacrylate copolymers quaternized with diethyl sulfate. Such compounds are obtainable commercially under the names Gafquat® 734 and Gafquat® 755,
vinylpyrrolidone/vinylimidazolium methochloride copolymers, such as those offered under the designations Luviquat® FC 370, FC 550, FC 905, and HM 552,
quaternized polyvinyl alcohol,
   and the polymers known by the names
Polyquatemium-2, Polyquaternium-17, Polyquaternium-18, Polyquaternium-24, Polyquaternium-27, Polyquaternium-32, Polyquatemium-37, Polyquaternium-74, and Polyquaternium-89.

Particularly preferred cationic polymers that can be included in the cosmetic carrier of method step f) are quaternized cellulose polymers, cationic guar derivatives, and/or cationic polymers based on acrylic acid (derivatives), which are selected in particular from the polymers known by the INCI names Guar Hydroxypropyltrimonium Chloride, Polyquaternium-6, Polyquaternium-7, Polyquatemium-10, Polyquatemium-37, and/or Polyquaternium-67.

The cationic care-providing and deposition polymer known by the INCI name Polyquaternium-7 is particularly preferred.

The cosmetic carriers described above can also include a number of further optional active agents that can produce advantageous properties on the skin and do not complicate the method according to the present invention. Included among the preferred optional active agents are, for example:
vitamins, vitamin derivatives, and/or vitamin precursors, which can be included in the cosmetic carrier of method step f) preferably in a quantity from 0.001 to 10 wt %, more preferably from 0.005 to 7.5 wt %, and in particular from 0.01 to 5 wt %, and/or
plant extracts and/or plant milks, which can be included in the cosmetic carrier of method step f) preferably in a quantity from 0.01 to 10 wt %, more preferably from 0.025 to 7.5 wt %, particularly preferably from 0.05 to 5 wt %, and in particular from 0.075 to 3 wt %.

Suitable vitamins are to be understood preferably as the following vitamins, provitamins, and vitamin precursors, as well as derivatives thereof:

Vitamin A: The group of substances referred to as “vitamin A” includes retinol (vitamin A₁) as well as 3,4-didehydroretinol (vitamin A₂). β-Carotene is the provitamin of retinol. Vitamin A components that are appropriate are, for example, vitamin A acid and esters thereof, vitamin A aldehyde, and vitamin A alcohol, as well as esters thereof such as the palmitate and acetate.

Vitamin B: Members of the vitamin B group or vitamin B complex are, among others:
Vitamin B₁ (thiamine)
Vitamin B₂ (riboflavin)
Vitamin B₃. The compounds nicotinic acid and nicotinic acid amide (niacinamide) are often listed under this designation.
Vitamin B₅ (pantothenic acid and panthenol). In the context of this group, panthenol is preferably used. Derivatives of panthenol that are usable are in particular esters and ethers of panthenol as well as cationically derivatized panthenols. Individual representatives are, for example, panthenol triacetate, panthenol monoethyl ether and the monoacetate thereof, and cationic panthenol derivatives.
Vitamin B₆ (pyridoxine as well as pyridoxamine and pyridoxal).

Vitamin C (ascorbic acid): Utilization in the form of the palmitic acid ester, glucosides, or phosphates can be preferred. Utilization in combination with tocopherols can likewise be preferred.

Vitamin E (tocopherols, in particular α-tocopherol).

Vitamin F. The term “vitamin F” is usually understood to mean essential fatty acids, in particular linoleic acid, linolenic acid, and arachidonic acid.

Vitamin H. “Vitamin H” refers to the compound (3aS,4S,6aR)-2-oxohexahydrothienol[3,4-d]-imidazole-4-valeric acid, for which the trivial name “biotin” has, however, now become established.

It is preferred to use vitamins, provitamins, and vitamin precursors from groups A, B, E, and H. Nicotinic acid amide, biotin, pantolactone, and/or panthenol are particularly preferred.

Suitable plant extracts and/or plant milks are to be understood as extracts that can be manufactured from all parts of a plant.

These extracts are usually produced by extraction of the entire plant. In individual cases, however, it may also be preferred to produce the extracts exclusively from blossoms and/or from leaves of the plant.

The extracts from green tea, oak bark, nettle, witch hazel, hops, chamomile, burdock root, horsetail, hawthorn, linden blossoms, lychee, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, ginseng, ginger root, Echinacea purpurea, Olea europea, Boerhavia diffusa roots, Foeniculum vulgaris, and Apium graveolens are especially suitable.

The extracts from green tea, stinging nettle, witch hazel, chamomile, aloe vera, ginseng, Echinacea purpurea, Olea europea, and/or Boerhavia diffusa roots are particularly preferred for use in the cleaning agents according to the present invention.

Water, alcohol, and mixtures thereof can be used as extraction agents for producing the aforesaid plant extracts. Among the alcohols, lower alcohols such as ethanol and isopropanol, but in particular polyvalent alcohols such as ethylene glycol and propylene glycol, both as the only extraction agent and mixed with water, are preferred. Plant extracts based on water/propylene glycol at a ratio from 1:10 to 10:1 have proven particularly suitable.

The plant extracts can be used in both pure and diluted form. If they are used in diluted form, they usually include approx. 2 to 80 wt % active substance, and include as a solvent the extraction agent or extraction agent mixture used to recover them.

Further active agents, adjuvants, and additives that can be used in the method according to the present invention are, for example:
humectants,
perfumes,
UV filters,
further thickening agents such as gelatin or plant gums, for example agar-agar, guar gum, alginates, xanthan gum, gum arabic, karaya gum, locust bean flour, linseed gums, dextrans, cellulose derivatives, e.g. methyl cellulose, hydroxyalkyl cellulose, and carboxymethyl cellulose, starch fractions and derivatives such as amylose, amylopectin, and dextrins, clays and sheet silicates such as bentonite, or entirely synthetic hydrocolloids such as polyvinyl alcohol, the calcium, magnesium, or zinc soaps,
structuring agents such as maleic acid and lactic acid,
dimethylisosorbide,
cyclodextrins,
fiber-structure-improving active agents, in particular mono-, di- and oligosaccharides such as glucose, galactose, fructose, fruit sugars, and lactose,
dyes for coloring the agent,
substances for adjusting pH, for example α- and β-hydroxycarboxylic acids as well as citric acid, lactic acid, malic acid, glycolic acid,
active agents such as bisabolol,
complexing agents such as EDTA, NTA, β-alaninediacetic acid, and phosphonic acids,
Ceramides “Ceramides” are understood as N-acylsphingosine (fatty acid amides of sphingosine) or synthetic analogs of such lipids (so-called pseudo-ceramides), propellants such as propane/butane mixtures, N₂O, dimethyl ether, CO₂, and air,
antioxidants,
consistency agents such as sugar esters, polyol esters, or polyolalkyl ethers,
preservatives, for example sodium benzoate and/or salicylic acid,
viscosity regulators such as salts (NaCl).

The method according to the present invention is preferably suitable for manufacturing conditioning cleaning agents that preferably have a pH in the region from 3 to 7, more preferably from 4 to 6.5, particularly preferably from 5 to 6.25, and in particular from 5.5 to 6.

Adjusting the cleaning agents to the pH range recited above has the advantage that additional preservation using parabens, etc. is not necessary.

The method according to the present invention has the advantage that it is particularly simple to carry out and requires little energy expenditure. The preparations of method steps a) and b), c) and d), and e) and f) can be mixed with one another in any sequence, and further adjuvants and active agents can be incorporated into the cosmetic carrier without thereby making the method appreciably more complex.

The resulting cleaning agents can include a high proportion of care-providing oil components (in the O/W emulsion) without negatively affecting the stability and foam properties of the agents.

The agents leave behind a soft, supple feel on the skin after being rinsed off.

A further advantage is that with the method according to the present invention it is possible to manufacture cosmetic cleaning agent having a distinctly lotion-like character, so that the care-providing effect of the cleaning agent can be visualized.

A second subject of the invention is a cosmetic cleaning agent that includes
0.01 to 5 wt % of at least one inorganic pigment and/or at least one opacifier and/or luster agent,
0.01 to 10 wt % of at least one acrylate homo- or copolymer,
0.1 to 15 wt % of at least one O/W emulsion that includes
(i) at least one fatty alcohol and/or at least one ethoxylated fatty alcohol of the following formula (I)

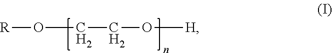

in which R denotes a straight-chain or branched, saturated or unsaturated alkyl residue having 10 to 22 carbon atoms, and the index n denotes the number 0 or a number from 5 to 25,
(ii) glycerol and/or at least one glycerol mono- and/or diester of glycerol and at least one straight-chain or branched, saturated or unsaturated carboxylic acid that has a carbon chain length from 10 to 22 carbon atoms,
(iii) at least one fatty acid ester of the following formula (II)

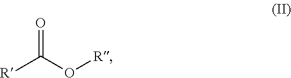

in which R′ denotes a straight-chain or branched, saturated or unsaturated alkyl residue having 5 to 18 carbon atoms, and R″ denotes a straight-chain or branched, saturated or unsaturated alkyl residue having 10 to 22 carbon atoms,
3 to 20 wt % of at least one anionic surfactant, and
0.1 to 15 wt % of at least one amphoteric, zwitterionic, or nonionic surfactant,
   wherein the quantity indications refer to the total weight of the cleaning agent.

The statements made regarding the method according to the present invention apply mutatis mutandis with respect to further preferred embodiments of the cleaning agent according to the present invention.

### EXAMPLES

1) Shower Cream and Lotion:

| | |
|---|---|
| Sodium Laureth Sulfate | 9.1 |
| Cocamidopropyl Betaine | 1.9 |
| Acrylates Copolymer | 1.65 |
| PEG-7 Glyceryl Cocoate | 0.8 |
| Perfume | 0.6 |
| Glycerin | 0.51 |
| Sodium Chloride | 0.45 |
| Sodium Benzoate | 0.4 |
| Styrene/Acrylates Copolymer | 0.4 |
| Citric Acid | 0.35 |
| Sodium Hydroxide | 0.34 |
| Polyquaternium-7 | 0.27 |
| Laureth-2 | 0.25 |
| Sodium Salicylate | 0.23 |
| Hydrogenated Castor Oil | 0.1 |
| Propylene Glycol | 0.1 |
| PEG-55 Propylene Glycol Oleate | 0.1 |
| Cetearyl Isononanoate | 0.075 |
| Ceteareth-20 | 0.04 |
| Cetearyl Alcohol | 0.04 |
| Glyceryl Stearate | 0.0125 |
| Panthenol | 0.0075 |
| Ceteareth-12 | 0.005 |
| Cetyl Palmitate | 0.005 |
| Water | to 100 |

2) Lustrous Shower Cream and Lotion:

| | |
|---|---|
| Sodium Laureth Sulfate | 9.5 |
| Cocamidopropyl Betaine | 1.9 |
| Acrylates Copolymer | 1.65 |
| PEG-7 Glyceryl Cocoate | 0.8 |
| Perfume | 0.6 |
| Glycerin | 0.5 |
| Sodium Chloride | 0.45 |
| Glycol Distearate | 0.45 |
| Sodium Benzoate | 0.4 |
| Styrene/Acrylates Copolymer | 0.4 |
| Citric Acid | 0.35 |
| Sodium Hydroxide | 0.34 |
| Polyquaternium-7 | 0.27 |
| Laureth-2 | 0.25 |
| Sodium Salicylate | 0.23 |
| Cocamide MEA | 0.225 |
| Hydrogenated Castor Oil | 0.1 |
| Propylene Glycol | 0.1 |
| PEG-55 Propylene Glycol Oleate | 0.1 |
| Laureth-10 | 0.1 |
| Cetearyl Isononanoate | 0.075 |
| Ceteareth-20 | 0.0375 |
| Cetearyl Alcohol | 0.0375 |
| Formic Acid | 0.015 |
| Glyceryl Stearate | 0.0125 |
| Panthenol | 0.0075 |
| Ceteareth-12 | 0.005 |
| Cetyl Palmitate | 0.005 |
| Water | to 100 |

The cosmetic cleaning agents of Examples 1 and 2 were manufactured using the following method:
a) providing an aqueous dispersion that includes Formic Acid, Laureth-10, Cocamide MEA, Glycol Distearate, Panthenol, Glycerin, Sodium Benzoate, Sodium Salicylate, Polyquaternium-7, Cocamidopropyl Betaine, Cetearyl Isononanoate, Ceteareth-20, Cetearyl Alcohol, Glyceryl Stearate, Ceteareth 12, Cetyl Palmitate, and Sodium Laureth Sulfate,
b) providing a preparation that includes Acrylates Copolymer, water, NaOH, and Sodium Laureth Sulfate,
c) mixing the aqueous dispersion from method step a) with the preparation from method step b),
d) providing an O/W emulsion of Hydrogenated Castor Oil, PEG-7 Glyceryl Cocoate, and water,
e) mixing the O/W emulsion from method step d) with the mixture from method step c),
f) providing a cosmetically acceptable carrier made up of the remaining ingredients, which includes inter alia water, Laureth-2, PEG-55 Propylene Glycol Oleate, and Propylene Glycol,
g) mixing the cosmetically acceptable carrier from method step f) with the mixture from method step e).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for manufacturing a cosmetic cleaning agent, which includes the following steps:
a) providing an aqueous dispersion that includes at least one inorganic pigment and/or at least one opacifier and/or luster agent,
b) providing a preparation that includes at least one acrylate homo- or copolymer,
c) mixing the aqueous dispersion from method step a) with the preparation from method step b),
d) providing an O/W emulsion,
e) mixing the O/W emulsion from method step d) with the mixture from method step c),
f) providing a cosmetically acceptable carrier that includes at least one surfactant,
g) mixing the cosmetically acceptable carrier from method step f) with the mixture from method step e).

2. The method according to claim 1, wherein the aqueous dispersion in method step a) includes 0.01 to 5 wt % of
at least one inorganic pigment having a refractive index >1.8,
at least one mono- and/or diester of ethylene glycol, of 1,2-propanediol, of glycerol, and/or of a polyethylene glycol with at least one C₈ to C₂₄ carboxylic acid, and/or
at least one styrene/acrylate copolymer,
wherein the quantity indications refer to the total weight of the cosmetic cleaning agent.

3. The method according to claim 1, wherein the acrylate homo- or copolymer is one or more polymers selected from the group consisting of crosslinked or uncrosslinked hydrophobically modified polyacrylates, and crosslinked or uncrosslinked copolymers of (meth)acrylic acid with at least one (meth)acrylic acid ester.

4. The method according to claim 1, wherein the O/W emulsion in method step d) includes, in addition to water,
(i) at least one fatty alcohol and/or at least one ethoxylated fatty alcohol of the following formula (I) 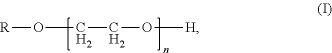
in which R denotes a straight-chain or branched, saturated or unsaturated alkyl residue having 10 to 22 carbon atoms, and the index n denotes the number 0 or a number from 5 to 25,
(ii) glycerol and/or at least one glycerol mono- and/or diester of glycerol and at least one straight-chain or branched, saturated or unsaturated carboxylic acid that has a carbon chain length from 10 to 22 carbon atoms, and
(iii) at least one fatty acid ester of the following formula (II) 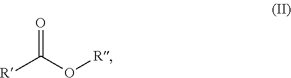
in which R′ denotes a straight-chain or branched, saturated or unsaturated alkyl residue having 5 to 18 carbon atoms, and R″ denotes a straight-chain or branched, saturated or unsaturated alkyl residue having 10 to 22 carbon atoms.

5. The method according to claim 4, wherein the O/W emulsion in method step d) includes
(i) at least one fatty alcohol and at least one ethoxylated fatty alcohol in accordance with formula (I),
(ii) glycerol and at least one glycerol mono- and/or diester, and
(iii) at least two different carboxylic acid esters of formula (II).

6. The method according to claim 1, wherein the O/W emulsion is used in a quantity from 0.1 to 20 wt %, the quantity indications referring to the total weight of the cleaning agent.

7. The method according to claim 1, wherein the cosmetic carrier in method step f) includes
a. 3 to 20 wt % of at least one anionic surfactant, and
b. 0.1 to 15 wt % of at least one amphoteric, zwitterionic, or nonionic surfactant,
wherein the quantity indications refer to the total weight of the cleaning agent.

8. The method according to claim 1, wherein the cosmetic carrier in method step f) additionally includes 0.01 to 20 wt % of at least one cationic polymer, the quantity indications referring to the total weight of the cleaning agent.

9. A cosmetic cleaning agent including:
0.01 to 5 wt % of at least one inorganic pigment and/or at least one opacifier and/or luster agent,
0.01 to 10 wt % of at least one acrylate homo- or copolymer,
0.1 to 15 wt % of at least one O/W emulsion that includes
(i) at least one fatty alcohol and/or at least one ethoxylated fatty alcohol of the following formula (I) 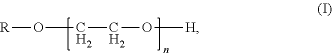
in which R denotes a straight-chain or branched, saturated or unsaturated alkyl residue having 10 to 22 carbon atoms, and the index n denotes the number 0 or a number from 5 to 25,
(ii) glycerol and/or at least one glycerol mono- and/or diester of glycerol and at least one straight-chain or branched, saturated or unsaturated carboxylic acid that has a carbon chain length from 10 to 22 carbon atoms,
(iii) at least one fatty acid ester of the following formula (II) 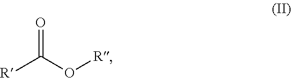
in which R′ denotes a straight-chain or branched, saturated or unsaturated alkyl residue having 5 to 18 carbon atoms, and R″ denotes a straight-chain or branched, saturated or unsaturated alkyl residue having 10 to 22 carbon atoms,
3 to 20 wt % of at least one anionic surfactant, and
0.1 to 15 wt % of at least one amphoteric, zwitterionic, or nonionic surfactant,
wherein the quantity indications refer to the total weight of the cleaning agent.
